(19) United States
(12) Patent Application Publication
(10) Pub. No.: US 2019/0108919 A1
(43) Pub. Date: Apr. 11, 2019

(54) TELEHEALTH DATA STORAGE SYSTEM

(71) Applicant: KONINKLIJKE PHILIPS N.V., EINDHOVEN (NL)

(72) Inventors: Kevin SHIM, Ellicott City, MD (US); Mark LINDELL, Cockeysville, MD (US)

(21) Appl. No.: 16/093,889

(22) PCT Filed: Apr. 26, 2017
(86) PCT No.: PCT/EP2017/059893

§ 371 Date: Oct. 15, 2018

Related U.S. Application Data
(60) Provisional application No. 62/327,572, filed on Apr. 26, 2016.

(51) Int. Cl. G16H 80/00; G16H 30/20; G06F 21/62

(52) U.S. Cl. CPC G16H 80/00; G06F 21/6245; G16H 30/20

(57) ABSTRACT

The present system relates to a telehealth system configured to store medical information associated with a subject. The system comprises one or more medical devices, first non-transient electronic storage, second non-transient electronic storage, a telehealth coordinator user interface, one or more hardware processors, and/or other components. The one or more medical devices are configured to generate the medical information. The medical information comprises protected health information (PHI) and non-protected health information (non-PHI). The first non-transient electronic storage is associated with a first entity. The first non-transient electronic storage is configured to store the PHI. The first non-transient electronic storage is one or both of: electronically protected by a firewall associated with the first entity; or located within an international boundary associated with the first entity. The hardware processors are configured by machine readable instructions to and aggregate the PHI and the non-PHI at the telehealth coordinator user interface.

### BACKGROUND

### 1. Field

The present disclosure pertains to a telehealth system and a method for storing medical information associated with a subject.

### 2. Description of the Related Art

Telehealth systems that provide point-to-point communication as a substitute for an in-person clinical visit are known. Typical prior art systems utilize electronic storage service providers to store patient data. These prior art systems often do not comply with governmental and/or regulatory medical information privacy requirements because the storage service providers do not store the electronic medical information as required by the regulations. For example, the electronic storage service providers may store the electronic medical information in data centers that may or may not be located in a physical location (e.g., within a specific country) and/or equipped with electronic security protections (e.g., behind an electronic firewall associated with a medical care provider) specified by the regulations.

### SUMMARY

Accordingly, one or more aspects of the present disclosure relate to a telehealth system configured to store medical information associated with a subject. The system comprises one or more medical devices, first non-transient electronic storage, second non-transient electronic storage, a telehealth coordinator user interface, one or more hardware processors, and/or other components. The one or more medical devices are configured to generate the medical information. The medical information comprises protected health information (PHI) and non-protected health information (non-PHI). The first non-transient electronic storage is associated with a first entity. The first non-transient electronic storage is configured to store the PHI. The first non-transient electronic storage is one or both of electronically protected by a firewall associated with the first entity; or located within an international boundary associated with the first entity. The second non-transient electronic storage is associated with a second entity. The second non-transient electronic storage is configured to store the non-PHI. The telehealth coordinator user interface is configured to provide the PHI and the non-PHI to a telehealth coordinator to facilitate healthcare management for the subject. The one or more hardware processors are configured by machine readable instructions to: obtain the medical information from the one or more medical devices; store the PHI in the first non-transient electronic storage; store the non-PHI in the second non-transient electronic storage; and aggregate the PHI and the non-PHI at the telehealth coordinator user interface.

Another aspect of the present disclosure relates to a method for storing medical information associated with a subject with a telehealth system. The system comprises one or more medical devices, first non-transient electronic storage, second non-transient electronic storage, a telehealth coordinator user interface, one or more hardware processors, and/or other components. The method comprises generating, with the one or more medical devices, the medical information. The medical information comprises protected health information (PHI) and non-protected health information (non-PHI). The method further comprises: obtaining, with the one or more hardware processors, the medical information from the one or more medical devices; and storing, with the one or more hardware processors, the PHI in the first non-transient electronic storage. The first non-transient electronic storage is associated with a first entity. The first non-transient electronic storage is one or both of electronically protected by a firewall associated with the first entity; or located within an international boundary associated with the first entity. The method further comprises storing, with the one or more hardware processors, the non-PHI in the second non-transient electronic storage, the second non-transient electronic storage associated with a second entity; aggregating, with the one or more hardware processors, the PHI and the non-PHI at the telehealth coordinator user interface; and providing, with the telehealth coordinator user interface, the PHI and the non-PHI to a telehealth coordinator to facilitate healthcare management for the subject.

Still another aspect of present disclosure relates to a telehealth system configured to store medical information associated with a subject. The system comprises means for generating the medical information. The medical information comprises protected health information (PHI) and non-protected health information (non-PHI). The system further comprises means for electronically storing the PHI. The means for electronically storing the PHI is associated with a first entity. The means for electronically storing the PHI is one or both of: electronically protected by a firewall associated with the first entity; or located within an international boundary associated with the first entity. The system further comprises means for electronically storing the non-PHI. The means for electronically storing the non-PHI is associated with a second entity. The system further comprises means for providing the PHI and the non-PHI to a telehealth coordinator to facilitate healthcare management for the subject; means for obtaining the medical information; and means for storing the PHI in the means for electronically storing the PHI and storing the non-PHI in the means for electronically storing the non-PHI; and means for aggregating the PHI and the non-PHI at the means for providing the PHI and non-PHI to the telehealth coordinator.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 is a schematic illustration of a system configured to store medical information associated with a subject.

FIG. 2 illustrates protected health information and non-protected health information flow between a telehealth coordinator user interface, a processor, non-transient electronic storage, and a vendor interface system.

FIG. 3 illustrates an example of technical diagram where the protected health information is stored and managed inside a firewall of a customer data center.

FIG. 4 illustrates an embodiment of the system where components of the system are located in two different countries.

FIG. 5 illustrates an embodiment of the system where components of the system are located in three different countries.

FIG. 6 illustrates an MPLS VPN connection between the premises of a medical device and/or other vendor and the premises of a customer.

FIG. 7 illustrates a method for storing medical information associated with a subject with a telehealth system.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

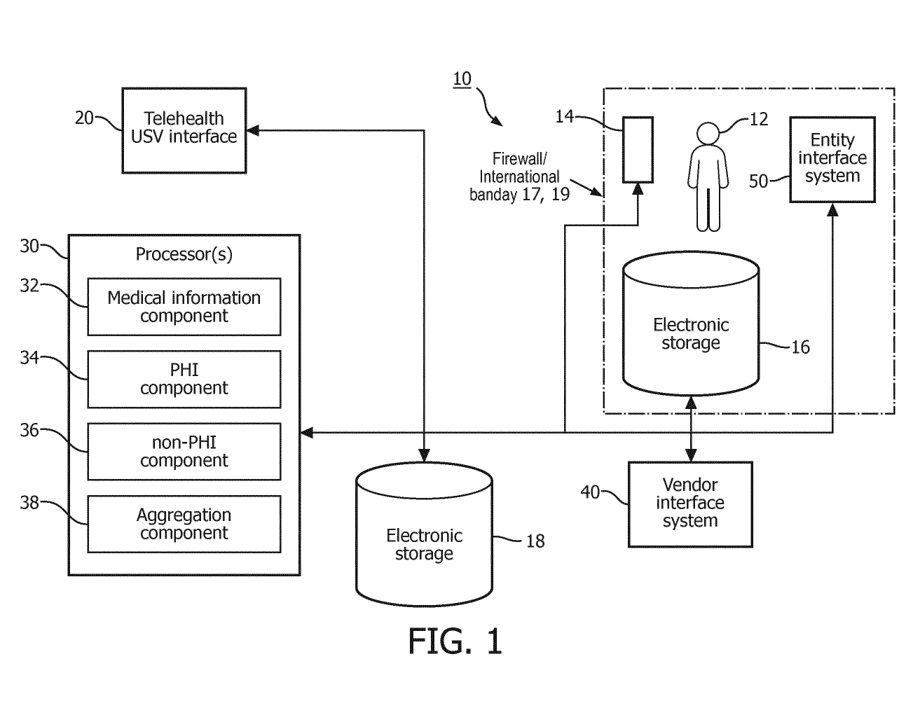

FIG. 1 is a schematic illustration of a telehealth system 10 configured to store medical information associated with a subject 12. System 10 is configured to facilitate coordinated health care for managing subjects across a care continuum (e.g., from hospital to home). System 10 is an integrated telehealth solution that helps reduce healthcare costs and improve subject outcomes. System 10 helps ensure care and access are delivered at the right place at the right time with the highest level of quality. System 10 leverages healthcare workforce skills by electronically bringing ill subjects into contact with the healthcare workforce through technology assisted remote care. System 10 is not anchored to specific sites, facilities, and/or institutions such as hospitals and/or doctor's offices. For example, system 10 facilitates communication between telehealth coordinators associated with a physical and/or virtual telehealth center with subjects, hospitals, caregivers (e.g., doctors, nurses, clinicians, family members, etc.), vendors, insurance providers, healthcare management providers, and/or other health service providers located throughout the world.

System 10 utilizes secure electronic connections to obtain and store private health information (PHI) and non-private health information (non-PHI) as required by applicable laws and/or regulations to enable enhanced collaboration between these users. For example, in some embodiments, system 10 is configured such that PHI resides in an electronic storage facility in a specific country where the laws and/or regulations specify the electronic storage facility must reside. In some embodiments, system 10 is configured such that PHI is protected by an electronic firewall and/or other electronic privacy protection measures associated with a healthcare management provider, hospital, doctor's office, etc. as the laws and/or regulations specify. System 10 is configured to comply with health information security requirements of various countries (e.g., including but not limited to the USA, Canada, Australia, Singapore, South Korea, China, the Netherlands, UK, Germany, France, and other European countries, etc.) by storing PHI in data centers within individual countries, on the premises of a customer (e.g., private health care management providers, public health care management providers such as ministries of health or veteran's affairs (VA) centers, individual hospitals, doctor's office, etc.) owned data center, behind an electronic firewall and/or other electronic privacy protection measures associated with the customer, and/or in other locations. In some embodiments, system 10 comprises one or more of medical devices 14, first electronic storage 16, second electronic storage 18, a telehealth coordinator user interface 20, a processor 30, a vendor interface system 40, an entity interface system 50, and/or other components.

Medical devices 14 are configured to generate the medical information. In some embodiments, medical devices 14 include but are not limited to equipment used in hospitals, doctor's offices, and/or other medical facilities to monitor vital signs and/or other physiological information (e.g., pulse rate monitors, blood pressure monitors, blood oxygenation monitors, glucose monitors, weight scales, thermometers, electrocardiogram (EKG) equipment, childbirth labor contraction monitors, etc.), test equipment (e.g., imaging equipment such as an MRI and/or an x-ray machine, an ultrasound, electroencephalogram (EEG) equipment, etc.), equipment for treating subject 12 (e.g., respirators/ventilators, light therapy devices, etc.), devices for entering and/or selecting information (e.g., desktop computers, laptop computers, tablet computers, smartphones, cameras, video equipment, etc.) associated with subject 12, and/or other devices. The medical information comprises protected health information (PHI), non-protected health information (non-PHI), and/or other information. The individual portions of the medical information that comprise the PHI and the non-PHI are determined at manufacture, programmed via telehealth coordinator user interface 20, determined by processor 30, and/or determined in other ways. For example, portions of the medical information that are PHI are pre-determined at manufacture of system 10 based on laws and/or regulations of a specific country and/or other governing body which specifies health information privacy rules. As another example, a telehealth coordinator may specify what portions of the medical information are PHI based on laws and/or regulations known to the telehealth coordinator. PHI includes any information that identifies an individual subject; information whose improper use and/or disclosure may adversely affect the ability of health care management providers, hospitals, doctor's offices, etc. to provide medical care; proprietary information; electronic records protected by confidentiality laws and/or regulations; and/or other information. For example, in some embodiments, the PHI includes a last name of subject 12, a first name of subject 12, a date of birth of subject 12, a last four digits of a social security number of subject 12, a residence address of subject 12, a gender of subject 12, an email address of subject 12, a phone number (e.g., mobile and/or home) of subject 12, a fax number of subject 12, a medical identification number associated with subject 12, an HL7 ID, a nickname of subject 12, a payer plan number associated with subject 12, physiological and/or other health information (e.g., diagnoses, test results, current health status and/or other health information) associated with subject 12, and/or other information. In some embodiments, one or more medical devices 14 are configured such that the medical information further comprises device information.

First non-transient electronic storage 16 is configured to store the PHI. First non-transient electronic storage 16 is associated with a first entity. The first entity comprises a country (e.g., Singapore), a medical facility (e.g., a hospital), a healthcare management provider (e.g., a veteran's affairs medical system, a ministry of health), another customer who receives services provided by system 10, and/or other entities. For example, first non-transient electronic storage may be an electronic data center associated with a public and/or private health care provider located in a specific country. The specific country may have laws and/or regulations that specify that PHI must be physically stored in a data center inside the specific country, the PHI must be electronically secured for privacy protection of patients (e.g., subject 12) by an associated medical facility and/or healthcare management provider, and/or have other requirements. As such, in some embodiments, first non-transient electronic storage 16 is one or both of electronically protected by a firewall 17 and/or other electronic privacy protection features associated with the first entity; and/or located within an international boundary 19 associated with the first entity. The physical location of first non-transient electronic storage 16 and/or the electronic privacy protection features of first non-transient electronic storage 16 depend on the nature of the first entity (e.g., whether the first entity is a country, a medical facility, a healthcare management provider, etc.), the laws and/or regulations associated with the first entity (e.g., which may require a certain type and/or level of privacy protection features), and/or other factors. It should be noted that the description of the location of first non-transient electronic storage 16 is not intended to be limiting. First non-transient electronic storage 16 may have any virtual and/or physical location and/or electronic privacy protection features that meet the laws and/or regulations associated with the first entity.

Second non-transient electronic storage 18 is configured to store the non-PHI. In some embodiments, second non-transient electronic storage is configured to store the device information and/or other non-PHI. Second non-transient electronic storage 18 is associated with a second entity. The second entity comprises a storage service provider, a telehealth system provider, a vendor, and/or other entities. For example, the second entity may include a storage service provider such as Salesforce.com and/or other storage service providers. In some embodiments, the non-PHI stored in second non-transient storage 18 is not subject to the storage location requirements and/or the information privacy requirements required for the PHI stored in first non-transient electronic storage 16. As such, unlike first non-transient electronic storage 16, second non-transient electronic storage 18 does not necessarily need to be electronically protected by the same firewall 17 and/or other electronic privacy protection features associated with the first entity (though it may have its own electronic security features), and/or second non-transient electronic storage 18 does not need to be located within the international boundary 19 associated with the first entity (though it may be located within international boundary 19 anyway). By way of a non-limiting example, for a first entity comprising the Singaporean Ministry of Health (e.g., a first entity that is a customer of the services provided by system 10), second non-transient electronic storage 18 may be located in the United States while first non-transient electronic storage 16 is located in Singapore because the laws of Singapore dictate that PHI from the Singaporean Ministry of Health be kept in Singapore.

It should be noted that the illustration of first and second non-transient electronic storage 16 and 18 as two separate devices is not intended to be limiting. In some embodiments, second non-transient electronic storage 18 is also associated with the first entity. In some embodiments, second non-transient electronic storage 18 may be physically included in first non-transient electronic storage 16 (e.g., such that second non-transient electronic storage 18 forms a portion of first non-transient electronic storage 16), and/or be included in the same data center that includes first non-transient storage 16, but remain outside of and/or unprotected by the firewall and/or other electronic privacy protections associated with the first entity and/or first non-transient storage 16 (e.g., so that information stored by second non-transient storage 18 is accessible by vendor interface system 40).

First non-transient electronic storage 16 and/or second non-transient electronic storage 18 comprise electronic storage media that electronically stores information. In some embodiments, first non-transient electronic storage 16 and/or second non-transient electronic storage 18 are and/or are included in data centers associated with the first and second entities respectively, and/or only the data center associated with the first entity. The electronic storage media of first non-transient electronic storage 16 and/or second non-transient electronic storage 18 may comprise system storage that is provided integrally (i.e., substantially non-removable) with system 10. For example, first non-transient electronic storage 16 may be and/or be included in a server located behind the firewall 17 and/or within the international boundary 19 associated with the first entity. Second non-transient electronic storage may be and/or be included in the first server and/or in a second separately located server, for example. In some embodiments, first non-transient electronic storage 16 and/or second non-transient electronic storage 18 may individually comprise a plurality of non-transient storage devices. For example, first non-transient electronic storage 16 may be and/or be included in multiple servers together with one or more processors 30 located behind the firewall 17 and/or within the international boundary 19 associated with the first entity. Some or all of second non-transient electronic storage 18 may be located in a server together with one or more processors 30, in a computing device associated with a telehealth coordinator together with telehealth user interface 20, and/or in other locations. First non-transient electronic storage 16 and/or second non-transient electronic storage 18 may comprise one or more of optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), and/or other electronically readable storage media. First non-transient electronic storage 16 and/or second non-transient electronic storage 18 may store software algorithms; information determined by processor 30; information received via medical devices 14, telehealth user interface 20, vendor interface system 40, and/or entity interface system 50, and/or external computing systems; and/or other information that enables system 10 to function as described herein.

Telehealth coordinator user interface 20 is configured to provide the PHI and the non-PHI to a telehealth coordinator to facilitate healthcare management for subject 12. Telehealth coordinators use coordinator user interface 20 to facilitate disease management control protocols, track associated vital signs and other subject-specific information captured from a subject (e.g., subject 12) at home, at a medical facility, and/or in other locations. Telehealth coordinator user interface 20 may be located anywhere in the world and electronically inside and/or outside the firewall associated with the first entity. Telehealth user interface 20 provides the PHI and non-PHI to a telehealth coordinator at that location responsive to a request for the information (described below) from the telehealth coordinator. In some embodiments, telehealth coordinator user interface 20 is configured to provide the PHI and the non-PHI to the telehealth coordinator via an application program interface (API) comprising a coordinator graphical user interface (GUI) displayed to the telehealth coordinator. In some embodiments, the API and/or coordinator GUI is and/or includes a web browsing interface configured to facilitate access to the internet. In some embodiments, telehealth coordinator user interface 20 includes one or more processors, databases, displays, and/or other computing components configured to facilitate the management of the healthcare of subject 12. Telehealth coordinator user interface 20 is configured to communicate with medical devices 14, processor 30, electronic storage 16, electronic storage 18, vendor interface system 40, entity interface system 50, and/or other components of system 10. By way of non-limiting example, telehealth user interface 20 may be and/or be included in one or more of a desktop computer, a laptop computer, a handheld computer, a tablet computing platform, a NetBook, a Smartphone, a gaming console, and/or other computing platforms.

Vendor interface system 40 is configured to facilitate management of non-PHI information such as medical device information associated with medical devices 14, billing invoices, and/or other non-PHI information. In some embodiments, vendor interface system 40 includes one or more processors, databases, displays, and/or other computing components configured to facilitate the management of the non-PHI. Medical device information associated with medical devices 14 includes device names of devices used by subject 12, device model numbers, an inventory of available devices for use by subject 12 and/or other subjects, a location of a particular medical device 14 (e.g., at a particular hospital, in a home of subject 12), maintenance schedules for individual medical devices 14, and/or other device information. Vendor interface system 40 is configured to communicate with medical devices 14, telehealth user interface 20, processor 30, electronic storage 18, entity interface system 50, and/or other components of system 10. By way of a non-limiting example, the device information stored in second non-transient electronic storage 18 is electronically accessible to vendor interface system 40. Vendor interface system 40 may communicate with telehealth user interface 20 to facilitate management of medical device 14 inventory data used to submit billing invoices to a client (e.g., a medical care provider) and/or store device information such as a serial number for device inventory management. In some embodiments, vendor interface system 40 may be and/or include one or more of a desktop computer, a laptop computer, a handheld computer, a tablet computing platform, a NetBook, a Smartphone, a gaming console, and/or other computing platforms.

In some embodiments, system 10 is configured such that a high-performance multiprotocol level switching (MPLS) site-to-site virtual private network (VPN) adhering to any applicable privacy and/or security standards is used to communicate non-PHI between vendor interface system 40 and medical devices 14, electronic storage 16, electronic storage 18, telehealth user interface 20, processor 30, entity interface system 50 and/or other components of system 10. In some embodiments, this may be a dedicated network for a specific customer (e.g., a country, a medical facility, a healthcare management provider, etc.) and electronically coupled to the customer firewall and/or other electronic privacy protection gateways. Among other features, the MPLS VPN communication link provides reliable throughput; available service level agreements (SLA) with network performance at speeds of 100 Mbps or higher (e.g., up to 10 Gbps); and dedicated secure point-to-point connections that link information systems, networks, and/or components of information and systems and networks of a single compartment, where the connections do not access the globally-addressable internet. The MPLS VPN links information systems, networks, and/or components of information and systems and networks of a customer (e.g., a country, a medical facility, a healthcare management provider, etc.) data center (e.g., first non-transient electronic storage 16) established through virtual private network technology utilizing security controls that are compliant with FIPS 140-2 and NIST 800-53 coupled with auditing and monitoring of the connections, provided that the connections do not also provide access to the globally-addressable Internet. The MPLS VPN provides communication links that are packet screened to ensure that only authorized traffic is permitted to flow between the interconnected customer (e.g., a country, a medical facility, a healthcare management provider, etc.) and non-customer (e.g., a medical device vendor) facility; and maintains the capability to isolate and/or temporarily deactivate any communication link in the event that suspicious activity is detected by a security system associated with the first entity, the second entity, the vendor and/or other users of system 10.

Entity interface system 50 is associated with the first entity. Entity interface system 50 is configured to provide information to and/or receive information from care providers and/or other users associated with private health care management providers, public health care management providers such as ministries of health in Singapore and/or veteran's affairs (VA) centers, individual hospitals, doctor's offices, subject 12, and/or other users. In some embodiments, entity interface system 50 includes one or more computing terminals, a phone system, an EHR (Electronic Health Record) system, an EMR (Electronic Medical Record) system, user authentication controls, a directory, data reporting features, video conferencing features, processors, databases, and/or other components. Entity interface system 50 is configured to communicate with medical devices 14, processor 30, electronic storage 16, electronic storage 18, vendor interface system 40, and/or other components of system 10. By way of non-limiting example, entity interface system 50 may be and/or include one or more computing devices such as servers, desktop computers, laptop computers, handheld computers, tablet computing platforms, NetBooks, Smartphones, gaming consoles, and/or other computing platforms.

Telehealth coordinator user interface 20, vendor interface system 40, and/or entity interface system 50 are configured to receive information from and/or provide information to one or more users (e.g., a telehealth coordinator, a caregiver associated with a health care provider, a user associated with a medical device 14 vendor, etc.) of system 10. Telehealth coordinator user interface 20, vendor interface system 40, and/or entity interface system 50 are configured to provide an interface between system 10 and the users through which the users may provide information to and receive information from system 10. This enables data, cues, results, and/or instructions and any other communicable items, collectively referred to as “information,” to be communicated between a user (e.g., a telehealth coordinator, a caregiver associated with a health care provider, a user associated with a medical device 14 vendor, etc.) and processor 30, and/or other components of system 10.

Examples of interface devices suitable for inclusion in telehealth coordinator user interface 20, vendor interface system 40, and/or entity interface system 50 comprise a display, a graphical user interface, a touchscreen, a keypad, buttons, speakers, a microphone, switches, a keyboard, knobs, levers, an indicator light, an audible alarm, a printer, a haptic feedback device, and/or other interface devices. In some embodiments, telehealth coordinator user interface 20, vendor interface system 40, and/or entity interface system 50 individually comprise a plurality of separate interfaces (e.g., system 10 may include multiple telehealth coordinator user interfaces 20, multiple vendor interface systems 40, and/or multiple entity interface systems 50). In some embodiments, telehealth coordinator user interface 20, vendor interface system 40, and/or entity interface system 50 comprise at least one interface that is provided integrally with processor 30 and/or other components of system 10.

It is to be understood that other communication techniques, either hard-wired or wireless, are also contemplated by the present disclosure as telehealth coordinator user interface 20, vendor interface system 40, and/or entity interface system 50. For example, the present disclosure contemplates that telehealth coordinator user interface 20, vendor interface system 40, and/or entity interface system 50 may be integrated with a removable storage interface. In this example, information may be loaded into system 10 from removable storage (e.g., a smart card, a flash drive, a removable disk, etc.) that enables the user(s) to customize the implementation of system 10. The removable storage may be and/or include removable storage that is removably connectable to system 10 via, for example, a port (e.g., a USB port, a firewire port, etc.) or a drive (e.g., a disk drive, etc.). Other exemplary input devices and techniques adapted for use with system 10 as user telehealth coordinator user interface 20, vendor interface system 40, and/or entity interface system 50 comprise, but are not limited to, an RS-232 port, RF link, an IR link, modem (telephone, cable or other). In short, any technique for communicating information with system 10 is contemplated by the present disclosure as telehealth coordinator user interface 20, vendor interface system 40, and/or entity interface system 50.

Processor 30 is configured to provide information processing capabilities in system 10. As such, processor 30 may comprise one or more of a digital processor, an analog processor, a digital circuit designed to process information, an analog circuit designed to process information, a state machine, and/or other mechanisms for electronically processing information. Although processor 30 is shown in FIG. 1 as a single entity, this is for illustrative purposes only. In some embodiments, processor 30 may comprise a plurality of processing units. These processing units may be physically located within the same device (e.g., a server), or processor 30 may represent processing functionality of a plurality of devices operating in coordination (e.g., multiple servers with one server behind firewall 17/boundary 19 and another server outside firewall 17/boundary 19, telehealth user interface 20, computing devices associated with the first (e.g., entity interface system 50) and/or second entity, a computing device associated with a vendor (e.g., vendor interface system 40) and/or other users, first and/or second non-transient electronic storage 16 and/or 18, and/or other devices.)

In some embodiments, processor 30, medical devices 14, first non-transient electronic storage 16, second non-transient electronic storage 18, telehealth user interface 20, vendor interface system 40, and/or entity interface system 50 may be operatively linked via one or more electronic communication links. For example, such electronic communication links may be established, at least in part, via a network such as the Internet, the MPLS VPN, and/or other networks. It will be appreciated that this is not intended to be limiting, and that the scope of this disclosure includes embodiments in which these components may be operatively linked via some other communication media. In some embodiments, processor 30 is configured to communicate with medical devices 14, first non-transient electronic storage 16, second non-transient electronic storage 18, telehealth user interface 20, vendor interface system 40, and/or entity interface system 50 according to a client/server architecture, a peer-to-peer architecture, and/or other architectures.

As shown in FIG. 1, processor 30 is configured via machine-readable instructions to execute one or more computer program components. The one or more computer program components may comprise one or more of a medical information component 32, a PHI component 34, a non-PHI component 36, an aggregation component 36, and/or other components. Processor 30 may be configured to execute components 32, 34, 36, and/or 38 by software; hardware; firmware; some combination of software, hardware, and/or firmware; and/or other mechanisms for configuring processing capabilities on processor 30.

It should be appreciated that although components 32, 34, 36, and 38 are illustrated in FIG. 1 as being co-located within a single processing unit, in embodiments in which processor 30 comprises multiple processing units, one or more of components 32, 34, 36, and/or 38 may be located remotely from the other components (e.g., one or more components may be located within a server while one or more other components are located within a computing system associated with entity interface system 50). The description of the functionality provided by the different components 32, 34, 36, and/or 38 described below is for illustrative purposes, and is not intended to be limiting, as any of components 32, 34, 36, and/or 38 may provide more or less functionality than is described. For example, one or more of components 32, 34, 36, and/or 38 may be eliminated, and some or all of its functionality may be provided by other components 32, 34, 36, and/or 38. As another example, processor 30 may be configured to execute one or more additional components that may perform some or all of the functionality attributed below to one of components 32, 34, 36, and/or 38.

Medical information component 32 is configured to obtain the medical information from one or more medical devices 14. In some embodiments, medical information component 32 is configured such that obtaining the medical information includes transforming vital signs and/or other physiological information (e.g., pulse rate, oxygen level, etc.) in output signals from medical devices 14 into a data format (e.g., JSON API) suitable for electronic storage. In some embodiments, medical information component 32 routes the medical information for storage (e.g., to PHI component 34 and/or non-PHI component 36) via an application programming interface (e.g., a RESTful API) associated with processor 30, telehealth user interface 20, entity interface system 50, and/or other computing devices. Medical information component 32 is configured to identify portions of the information that are PHI and portions of the information that are non-PHI. Medical information component 32 is configured such that PHI includes any information that identifies an individual subject; information whose improper use and/or disclosure may adversely affect the ability of health care management providers, hospitals, doctor's offices, etc. to provide medical care; proprietary information; electronic records protected by confidentiality laws and/or regulations; and/or other information. For example, as described above, the PHI includes a last name of subject 12, a first name of subject 12, a date of birth of subject 12, a residence address of subject 12, an email address of subject 12, a phone number of subject 12, a medical identification number associated with subject 12, a payer plan number associated with subject 12, personal health information associated with subject 12, and/or other information.

Medical information component 32 is configured to separate the PHI portions of the information from the non-PHI portions of the information for storage. In some embodiments, medical information component 32 is configured to separate the portions of the medical information that comprise the PHI from the non-PHI portions based on predetermined instructions programmed at manufacture of system 10, programmed via telehealth coordinator user interface 20, determined by processor 30, and/or determined in other ways. Medical information component 32 is configured such that these predetermined instructions reflect the laws and/or regulations of a specific country and/or other governing body which specifies health information privacy rules.

PHI component 34 is configured to store the PHI in first non-transient electronic storage 16. In some embodiments, PHI component 34 is configured to encrypt the PHI for storage in first non-transient electronic storage 16. PHI component 34 and/or first non-transient electronic storage 16 are configured such that the PHI data is stored within the border of the first entity, inside the firewall and/or other electronic privacy protection features associated with the first entity, and/or in other locations. In some embodiments, storing the PHI in first non-transient electronic storage 16 includes electronically causing transmittal of the information identified as PHI and separated by medical information component 32 to first non-transient electronic storage 16.

Non-PHI component 36 is configured to store the non-PHI in second non-transient electronic storage 18. In some embodiments, this includes storing the device information in second non-transient electronic storage 18. In some embodiments, storing the non-PHI in second non-transient electronic storage 18 includes electronically causing transmittal of the information identified as non-PHI and separated by medical information component 32 from processor 30 to second non-transient electronic storage 18.

Aggregation component 38 is configured to aggregate the PHI and the non-PHI at telehealth coordinator user interface 20, entity interface system 50, and/or other interfaces. In some embodiments, aggregation component 38 is configured to aggregate the device information with the PHI and the non-PHI at telehealth coordinator user interface 20, entity interface system 50, and/or other interfaces via a web browser and/or other graphical user interfaces displayed to users via telehealth coordinator user interface 20, entity interface system 50, and/or other interfaces. Aggregation component 38 is configured such that only the non-PHI data such as device inventory information and/or billing information is accessible via vendor interface system 40. In some embodiments, aggregating the PHI and the non-PHI comprises obtaining the PHI from first non-transient electronic storage 16, obtaining the non-PHI from second non-transient electronic storage 18, and causing the display of the PHI and the non-PHI together in one or more views of a graphical user interface (e.g., a web browser) on telehealth coordinator user interface 20, entity interface system 50, and/or other interfaces. In some embodiments, aggregation component 38 aggregates the PHI and the non-PHI responsive to a request from a user (e.g., a telehealth coordinator, a caregiver, etc.) made via the graphical user interface displayed by telehealth coordinator user interface 20 and/or entity interface system 50. In some embodiments, the request may specify that the entirety of the PHI and/or non-PHI should be aggregated, a portion of the PHI and/or non-PHI should be aggregated (e.g., information generated during a previous visit to a doctor's office), specific elements of the PHI and/or non-PHI should be aggregated (e.g., a name, treatment codes, and a billing invoice number), and/or the request may specify other information.

In some embodiments, aggregation component 38 is configured such that aggregation includes causing display of the PHI and/or non-PHI in one or more fields of one or more views of the graphical user interface. For example, PHI including a last name of subject 12, a first name of subject 12, a date of birth of subject 12, a residence address of subject 12, an email address of subject 12, a phone number of subject 12, a medical identification number associated with subject 12, a payer plan number associated with subject 12, and/or personal health information reflecting a current health status (e.g., most recent heart rate, blood pressure, cholesterol level, and weight) of subject 12 may be obtained from first non-transient electronic storage 16. Non-PHI such as serial and/or model numbers of equipment used by subject 12 may be obtained from second non-transient electronic storage 18. Aggregation component 38 may cause the graphical user interface to display these individual pieces of information in one or more individual fields of one or more views of the graphical user interface. In some embodiments, aggregation component 38 is configured to facilitate video conferences in one or more fields of the graphical user interface between subject 12, users associated with the first entity (e.g., doctors), vendors, telehealth coordinators, and/or other users.

In some embodiments, aggregation component 38 is configured to aggregate the PHI and the non-PHI responsive to authentication by an authorized user (e.g., a telehealth coordinator, a caregiver, etc.) Aggregation component 38 may control the GUI to facilitate authentication by the authorized user by causing presentation of one or more authentication fields in the GUI and receiving authentication entries (e.g., fingerprints, authentication codes, etc.) and/or selections by the authorized user (e.g., answering security questions, etc.).

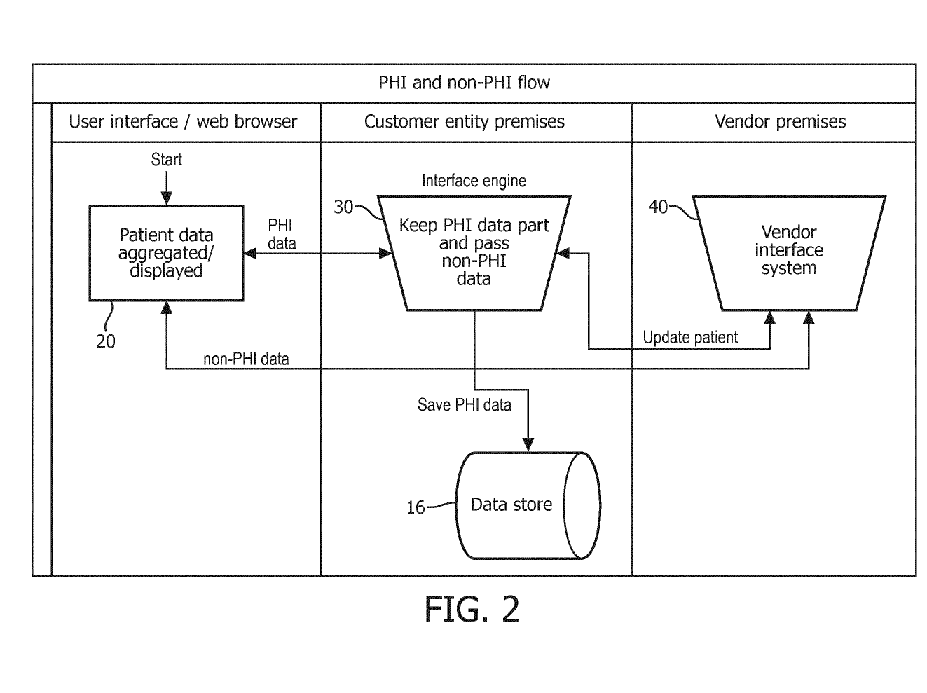

FIG. 2-5 illustrate various example embodiments of system 10. For example, FIG. 2 illustrates PHI and non-PHI flow between telehealth user interface 20, processor 30, first and second non-transient electronic storage 16 and 18, and vendor interface system 40. PHI and non-PHI is aggregated (e.g. responsive to a request from a telehealth coordinator) at telehealth user interface 20. The PHI data from first non-transient electronic storage 16 (located on the premises of the customer entity) is passed by processor 30 to interface 20. In this example, non-PHI passes directly between vendor interface system 40 and interface 20 (e.g., second non-transient electronic storage 18 is included in vendor interface system 40).

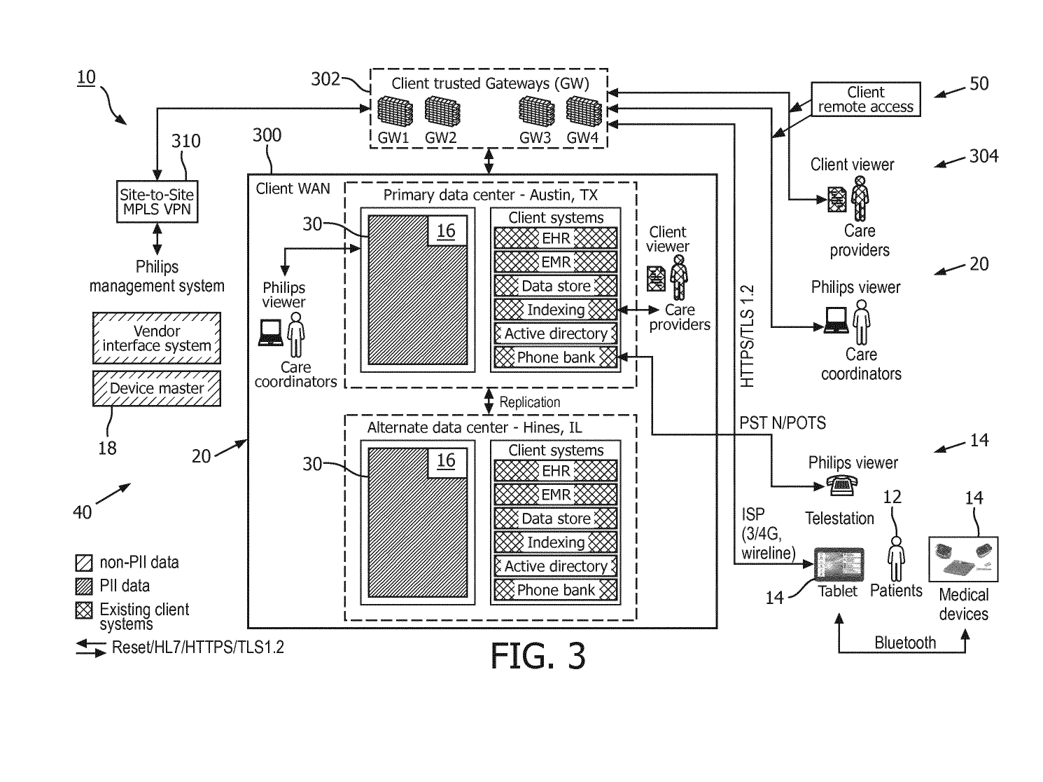

FIG. 3 is an example of a technical diagram where the PHI is stored and managed inside the firewall 300 of a customer (e.g., the first entity is the healthcare providing portion of the client data center). FIG. 3 illustrates subject 12, medical devices 14, two replicated (one replication located in Austin Tex. and the other replication in Hines Ill.) versions of processor 30 and first non-transient electronic storage 16 coupled with portions (EHR, EMR, Data Store, Indexing, active directory, and a phone bank) of the client system (entity interface system 50), telehealth user interfaces 20, vendor interface system 40 (which communicates with entity interface system 50 by way of a site-to-site MPLS VPN 310) including second non-transient electronic storage 18, and other components. In FIG. 3, entity interface system 50 comprises various gateways 302 (e.g., electronic links to entity interface system 50), viewers 304 (e.g., computing platforms associated with care providers), and other components. In FIG. 3, REST/HL7/HTTPS/TLS1.2 stands for Representational State Transfer/Health Level-7/Hypertext Transfer Protocol for Secure communication/Transport Layer Security. WAN stands for Wide Area Network.

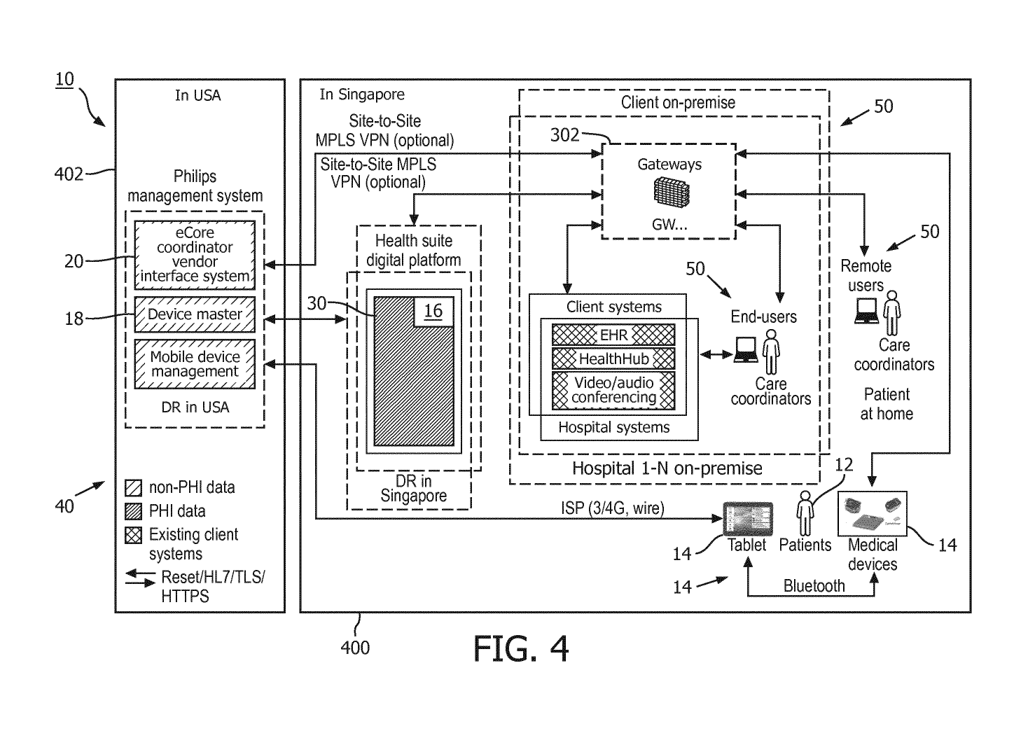

FIG. 4 illustrates an embodiment of system 10 where components of system 10 are located in two different countries, Singapore and the USA. In FIG. 4, the first entity is the Singaporean Ministry of Health Holdings (MOHH) as an example. As shown in FIG. 4, subject 12, medical devices 14, first non-transient electronic storage 16, processor 30, and entity interface system 50 are located in Singapore (e.g., within international boundary 400). Second non-transient electronic storage 18, telehealth user interface 20, and vendor interface system 40 are located in the USA (e.g., within international boundary 402). In FIG. 4, DR stands for Disaster Recovery. DC stands for data center.

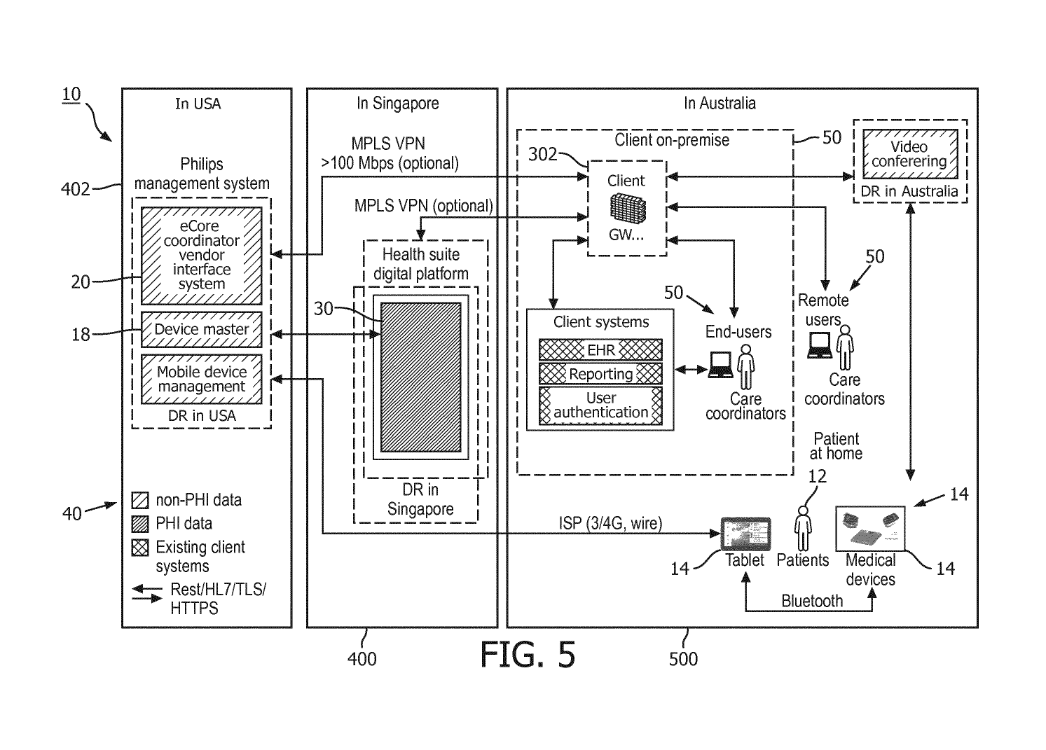

FIG. 5 illustrates an embodiment of system 10 where components of system 10 are located in three different countries, Australia, Singapore, and the USA. In FIG. 5, the first entity is an Australian Health Provider. As shown in FIG. 5, subject 12, medical devices 14, and entity interface system 50 are located in Australia (e.g., within international boundary 500). Processor 30 is located in Singapore (e.g., within international boundary 400). Second non-transient electronic storage 18, telehealth user interface 20, and vendor interface system 40 (not shown in FIG. 5) are located in the USA (e.g., within international boundary 402). In the example shown in FIG. 5 (and/or the other examples shown in other figures), system 10 may rely on identity based encryption (IBE) to protect the PHI.

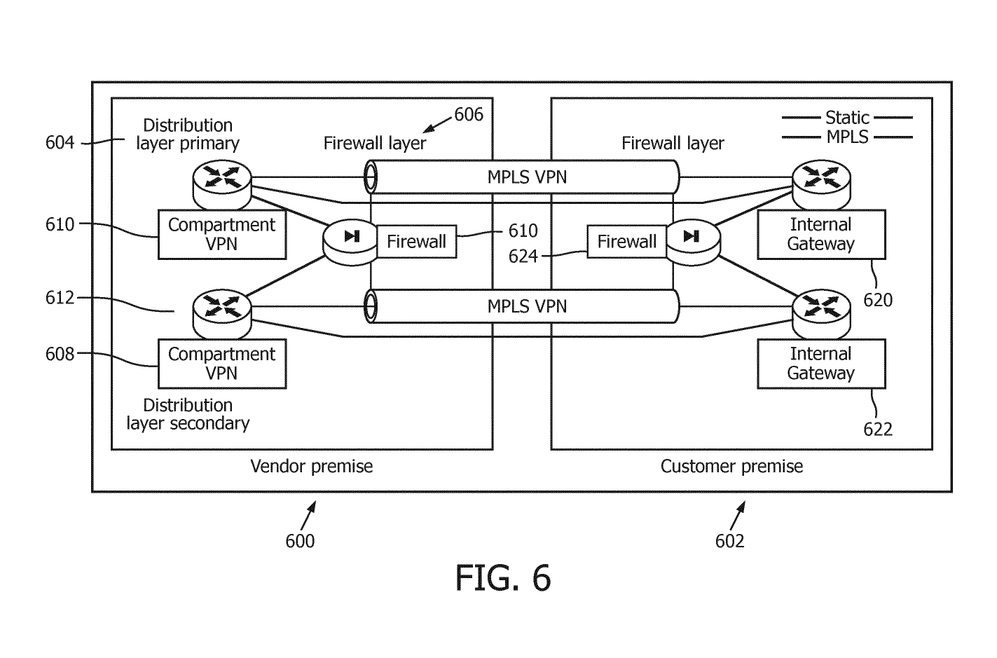

FIG. 6 illustrates the MPLS VPN connection between the premises 600 of a medical device and/or other vendor and the premises 602 of a customer (e.g., the first entity (as described above) in this example is the client). As shown in FIG. 6, on vendor premises 600, there is a primary distribution layer 604, a firewall layer 606, and a secondary distribution layer 608. There are also compartment VPNs 610 and 612 and a firewall 614. On customer premises 602, there are internal gateways 620 and 622, and a firewall 624.

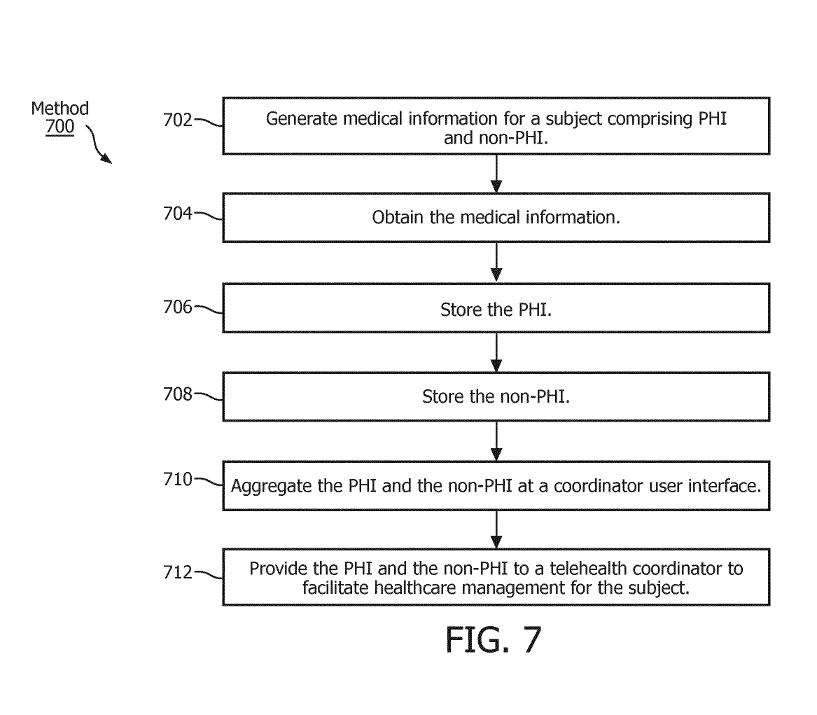

FIG. 7 illustrates a method 700 for storing medical information associated with a subject with a telehealth system. The system comprises one or more medical devices, first non-transient electronic storage, second non-transient electronic storage, a telehealth coordinator user interface, one or more hardware processors, and/or other components. The one or more hardware processors are configured by machine readable instructions to execute computer program components. The computer program components include a medical information component, a PHI component, a non-PHI component, an aggregation component, and/or other components. The operations of method 700 presented below are intended to be illustrative. In some embodiments, method 700 may be accomplished with one or more additional operations not described, and/or without one or more of the operations discussed. Additionally, the order in which the operations of method 700 are illustrated in FIG. 7 and described below is not intended to be limiting.

In some embodiments, method 700 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 700 in response to instructions stored electronically on an electronic storage medium. The one or more processing devices may include one or more devices configured through hardware, firmware, and/or software to be specifically designed for execution of one or more of the operations of method 700.

At an operation 702, medical information is generated. The medical information comprises PHI and non-PHI. In some embodiments, the PHI includes a last name of the subject, a first name of the subject, a date of birth of the subject, a residence address of the subject, an email address of the subject, a phone number of the subject, a medical identification number associated with the subject, and a payer plan number associated with the subject. In some embodiments, operation 702 is performed by medical devices the same as or similar to medical devices 14 (shown in FIG. 1 and described herein).

At an operation 704, the medical information is obtained from the medical devices. In some embodiments, operation 704 is performed by a processor component the same as or similar to medical information component 32 (shown in FIG. 1 and described herein).

At an operation 706, the PHI is stored in the first non-transient electronic storage. The first non-transient electronic storage is associated with a first entity. The first non-transient electronic storage is one or both of: electronically protected by a firewall associated with the first entity; or located within an international boundary associated with the first entity. In some embodiments, the first entity comprises one or more of a country, a medical facility, or a healthcare management provider. In some embodiments, operation 706 is performed by a processor component the same as or similar to PHI component 34 (shown in FIG. 1 and described herein).

At an operation 708, the non-PHI is stored in the second non-transient electronic storage. The second non-transient electronic storage is associated with a second entity. In some embodiments, the second entity comprises a storage service provider. In some embodiments, the second non-transient electronic storage is neither electronically protected by the firewall associated with the first entity or located within the international boundary associated with the first entity. In some embodiments, operation 708 is performed by a processor component the same as or similar to non-PHI component 36 (shown in FIG. 1 and described herein).

At an operation 710, the PHI and the non-PHI are aggregated at a telehealth coordinator user interface. In some embodiments, operation 710 is performed by a processor component the same as or similar to aggregation component 38 (shown in FIG. 1 and described herein).

At an operation 712, the aggregated PHI and non-PHI are provided to a telehealth coordinator. The aggregated PHI and non-PHI are provided via the telehealth coordinator user interface. In some embodiments, operation 712 is performed by an interface the same as or similar to telehealth coordinator user interface 20 (shown in FIG. 1 and described herein).

In some embodiments, the medical information further comprises device information and the method further comprises storing, with the one or more hardware processors, the device information in the second non-transient electronic storage such that the device information is electronically accessible to a device vendor interface system. In some embodiments, the method further comprises aggregating, with the one or more hardware processors, the device information with the PHI and the non-PHI at the telehealth coordinator user interface.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

1. A telehealth system configured to store medical information associated with a subject, the system comprising:
one or more medical devices configured to generate the medical information, the medical information comprising protected health information (PHI) and non-protected health information (non-PHI);
first non-transient electronic storage associated with a first entity, the first non-transient electronic storage configured to store the PHI, the first non-transient electronic storage being one or both of:
(1) electronically protected by a firewall associated with the first entity; or
(2) located within an international boundary associated with the first entity;
second non-transient electronic storage associated with a second entity, the second non-transient electronic storage configured to store the non-PHI;
a telehealth coordinator user interface configured to provide the PHI and the non-PHI to a telehealth coordinator to facilitate healthcare management for the subject; and
one or more hardware processors configured by machine readable instructions to:
obtain the medical information from the one or more medical devices;
store the PHI in the first non-transient electronic storage;
store the non-PHI in the second non-transient electronic storage; and
aggregate the PHI and the non-PHI at the telehealth coordinator user interface.

2. The system of claim 1, wherein the first entity comprises one or more of a country, a medical facility, or a healthcare management provider; and wherein the second entity comprises a storage service provider.

3. The system of claim 1, wherein the second non-transient electronic storage is neither electronically protected by the firewall associated with the first entity or located within the international boundary associated with the first entity.

4. The system of claim 1, wherein the one or more medical devices are configured such that the medical information further comprises device information, wherein the one or more hardware processors are configured to store the device information in the second non-transient electronic storage such that the device information is electronically accessible to a device vendor interface system, and wherein the one or more hardware processors are configured to aggregate the device information with the PHI and the non-PHI at the telehealth coordinator user interface.

5. The system of claim 1, wherein the PHI includes a last name of the subject, a first name of the subject, a date of birth of the subject, a last four digits of a social security number of the subject, a residence address of the subject, a gender of the subject, an email address of the subject, a home phone number and/or a mobile phone number of the subject, a fax number of the subject, a medical identification number associated with the subject, an HL7 ID, a nickname of the subject, and a payer plan number associated with the subject.

6. A method for storing medical information associated with a subject with a telehealth system, the system comprising one or more medical devices, first non-transient electronic storage, second non-transient electronic storage, a telehealth coordinator user interface, and one or more hardware processors, the method comprising:
generating, with the one or more medical devices, the medical information, the medical information comprising protected health information (PHI) and non-protected health information (non-PHI);
obtaining, with the one or more hardware processors, the medical information from the one or more medical devices;
storing, with the one or more hardware processors, the PHI in the first non-transient electronic storage, the first non-transient electronic storage associated with a first entity, the first non-transient electronic storage being one or both of:
(1) electronically protected by a firewall associated with the first entity; or
(2) located within an international boundary associated with the first entity;
storing, with the one or more hardware processors, the non-PHI in the second non-transient electronic storage, the second non-transient electronic storage associated with a second entity;
aggregating, with the one or more hardware processors, the PHI and the non-PHI at the telehealth coordinator user interface; and
providing, with the telehealth coordinator user interface, the PHI and the non-PHI to a telehealth coordinator to facilitate healthcare management for the subject.

7. The method of claim 6, wherein the first entity comprises one or more of a country, a medical facility, or a healthcare management provider; and wherein the second entity comprises a storage service provider.

8. The method of claim 6, wherein the second non-transient electronic storage is neither electronically protected by the firewall associated with the first entity or located within the international boundary associated with the first entity.

9. The method of claim 6, wherein the medical information further comprises device information, wherein the method further comprises storing, with the one or more hardware processors, the device information in the second non-transient electronic storage such that the device information is electronically accessible to a device vendor interface system, and wherein the method further comprises aggregating, with the one or more hardware processors, the device information with the PHI and the non-PHI at the telehealth coordinator user interface.

10. The method of claim 6, wherein the PHI includes a last name of the subject, a first name of the subject, a date of birth of the subject, a last four digits of a social security number of the subject, a residence address of the subject, a gender of the subject, an email address of the subject, a home and/or mobile phone number of the subject, a fax number of the subject, a medical identification number associated with the subject, an HL7 ID, a nickname of the subject, and a payer plan number associated with the subject.

11. A telehealth system configured to store medical information associated with a subject, the system comprising:
means for generating the medical information, the medical information comprising protected health information (PHI) and non-protected health information (non-PHI);
means for electronically storing the PHI, the means for electronically storing the PHI associated with a first entity, the means for electronically storing the PHI being one or both of:
(1) electronically protected by a firewall associated with the first entity; or
(2) located within an international boundary associated with the first entity;
means for electronically storing the non-PHI, the means for electronically storing the non-PHI associated with a second entity;
means for providing the PHI and the non-PHI to a telehealth coordinator to facilitate healthcare management for the subject;
means for obtaining the medical information;
means for storing the PHI in the means for electronically storing the PHI and storing the non-PHI in the means for electronically storing the non-PHI; and
means for aggregating the PHI and the non-PHI at the means for providing the PHI and non-PHI to the telehealth coordinator.

12. The system of claim 11, wherein the first entity comprises one or more of a country, a medical facility, or a healthcare management provider; and wherein the second entity comprises a storage service provider.

13. The system of claim 11, wherein the means for electronically storing the non-PHI is neither electronically protected by the firewall associated with the first entity or located within the international boundary associated with the first entity.

14. The system of claim 11, wherein the medical information further comprises device information, wherein the device information is stored in the means for electronically storing the non-PHI such that the device information is electronically accessible to a device vendor interface system, and wherein the device information is aggregated with the PHI and the non-PHI.

15. The system of claim 11, wherein the PHI includes a last name of the subject, a first name of the subject, a date of birth of the subject, a last four digits of a social security number of the subject, a residence address of the subject, a gender of the subject, an email address of the subject, a mobile and/or home phone number of the subject, a fax number of the subject, a medical identification number associated with the subject, an HL7 ID, a nickname of the subject, and a payer plan number associated with the subject.

16. A telehealth system configured to store medical information associated with a subject, the system comprising one or more hardware processors configured by machine readable instructions to:
obtain medical information from one or more medical devices configured to generate the medical information, the medical information comprising protected health information (PHI) and non-protected health information (non-PHI);
store, in first non-transient electronic storage associated with a first entity, the PHI, the first non-transient electronic storage being one or both of:
(1) electronically protected by a firewall associated with the first entity; or
(2) located within an international boundary associated with the first entity;
store, in second non-transient electronic storage associated with a second entity, the non-PHI; and
aggregate the PHI and the non-PHI at a telehealth coordinator user interface configured to provide the PHI and the non-PHI to a telehealth coordinator to facilitate healthcare management for the subject.
